(19) United States
(12) Patent Application Publication
(10) Pub. No.: US 2007/0178482 A1
(43) Pub. Date: Aug. 2, 2007

(54) METHOD FOR PREPARING SINGLE-STRANDED DNA

(75) Inventors: Alexander Lezhava, Kanagawa (JP); Yuko Shibata, Kanagawa (JP); Matthias Harbers, Kanagawa (JP); Toshizo Hayashi, Kanagawa (JP); Yoshihide Hayashizaki, Ibaraki (JP); Piero Carninci, Saitama (JP)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignees: KABUSHIKI KAISHA DNAFORM; RIKEN

(21) Appl. No.: 11/462,939

(22) Filed: Aug. 7, 2006

Related U.S. Application Data
(63) Continuation of application No. PCT/JP05/02097, filed on Feb. 4, 2005.

(30) Foreign Application Priority Data
Feb. 6, 2004 (JP) 2004-030686

(51) Int. Cl. C12Q 1/68; C12P 19/34

(52) U.S. Cl. 435/6; 435/91.2

(57) ABSTRACT

The invention is a method and a kit for preparing single-stranded DNA from double-stranded DNA and the purification of single-stranded DNA derived from double-stranded DNA. A single-stranded-DNA binding substance is used in combination with a double-strand-specific endonuclease for the removal of undesired double-stranded DNA from a single-stranded DNA preparation and for other related purposes.

### CROSS-REFERENCE TO RELATED APPLICATIONS

This is a continuation application of PCT/JP2005/002097, filed Feb. 4, 2005, which is incorporated herein by reference in its entirety, and also claims the benefit of Japanese Application No. 2004-030686, filed Feb. 6, 2004.

### FIELD OF THE INVENTION

The present invention relates to a method for preparing single-stranded DNA from double-stranded DNA and the purification of single-stranded DNA derived from double-stranded DNA. The present invention also relates to the removal of undesired double-stranded DNA from a single-stranded DNA preparation. Further, the present invention relates to a kit for the above-mentioned methods and applications involving prepared or purified single-stranded DNA.

### BACKGROUND ART

The genetic information of living organisms is stored in the form of double-stranded deoxyribonucleic acid (DNA), in which the sequences of the two strands are complementary to each other and associated by specific hydrogen bonds formed between individual base pairs in the sequences. For its analysis, manipulation or any other application in biotechnology or molecular biology, genomic DNA is usually fragmentized and cloned into a vector for its amplification and manipulation. Similarly transcribed regions of genomic DNA can be cloned by converting a transcript which is obtained in the form of a ribonucleic acid (RNA) into DNA by means of a reverse transcriptase. Standard technologies for the cloning and manipulation of DNA and RNA as known to those skilled in the art of molecular biology are described by J. Sambrook and D. W. Russell in “Molecular Cloning,” Cold Spring Harbor Laboratory Press, 2001, which is hereby incorporated herein by reference.

Most frequently, DNA is cloned into extrachromosomal molecules called plasmids, which are commonly composed of double-stranded DNA molecules covalently closed to form circular DNA molecules. Plasmids behave as accessory genetic units harboring regulatory elements in the so-called replicon and use the replication machinery of their host bacteria to maintain and control their copy numbers within the host cell. Often plasmids further contain genes encoding for enzymatic activities which can be used as selection markers. For the purpose of using plasmids as vectors for handling, amplifying and manipulating cloned DNAs, those selection markers commonly encode for genes conferring resistance to specific antibiotics, and thus allow for their selection by bacterial phenotypes.

Though the double-stranded form is the most commonly used DNA molecules, many applications and technologies in molecular biology and biotechnology require the strand-specific preparation of single-stranded DNA. Such applications include, but are not limited to, the preparation of a template DNA for sequencing or for strand-specific DNA synthesis including synthesis of labeled probes, the replacement of thymine residues by uracil, the introduction of point mutations, the preparation of testers and drivers for subtractive hybridizations or the detection and isolation of individual clones in a mixture of various DNA or RNA molecules, the detection and analysis of single nucleotide polymorphisms (SNPs), and the preparation of microarrays. Those methods and their applications are well known to those skilled in the art of molecular biology and are further described by J. Sambrook and D. W. Russell, ibid.

Standard approaches for the preparation of single-stranded DNA most frequently make use of so-called phagemids, which are plasmid-phage hybrids obtained by cloning the cis-acting regulatory sequences for the initiation and termination of DNA synthesis from the single-stranded genomic DNA of the bacteriophage M13 genome into cloning vectors. Such phagemids allow for the in vivo preparation of single-stranded DNA when the host bacteria are infected by a helper wild-type or mutant filamentous bacteriophage carrying replication-defective intergenic regions. After infection the gene II product encoded by the helper phage introduces a strand-specific nick into the intergenic region of the phagemids initiating a rolling-circle like replication of one strand. Thereafter, single-stranded copies of the phagemid DNA are packed into the progeny bacteriophage particles and extruded into the medium, from which the single-stranded DNA can be isolated. However, commonly used in the laboratory routines, the approach is limited to the use of a certain set of bacteria expressing a sex pili encoded by an F factor, and the use of phagemids harboring cis-acting elements from the bacteriophage M13 or related phages. Furthermore, the single-stranded DNA obtained by means of this approach is most often contaminated by helper phage DNA, small amounts of large chromosomal DNA and some RNA from the cell lysis. For many applications, therefore, a tedious and complicated purification of the single-stranded DNA is required to allow for its use. As an alternative to the in vivo preparation of single-stranded DNA, in vitro approaches have been developed making use of combinations of two different enzymatic activities. Most commonly, a combination of the replication initiator protein Gene II of the bacteriophage f1 and the exonuclease III from E. coli (ExoIII) is used in such systems. Here, Gene II will act as a site-specific endonuclease that recognizes the f1 ori in a phagemid vectors, and cleaves the viral strand. ExoIII attacks the free 3′-end of the nicked strand and digests it until the other strand is released as single-stranded circular DNA. Such a system can be commercially obtained e.g. as part of the so-called GeneTrapper® cDNA Positive Selection System from Gibco BRL/Life Technologies (CAT. NO. 10356-020, which is now part of Invitrogen Corporation, Carlsbad, USA). The Instruction Manual of this commercially available system is hereby incorporated herein by reference. As the efficiency of Gene II enzymatic activity in these reactions tends to be low, in some cases as low as yielding only in about 50% of the target DNA being cut, other strand-specific nicking enzymes have been developed. These include artificially engineered nicking endonucleases which cleave only one DNA strand within their recognition sequence on a double-stranded DNA substrate. Such enzymes include, but are not limited to, the commercially available nucleases N.Bpu 10I (FERMENTAS UAB, Vilnius, Lithuania), N.Bbv C IA, N.Bst NB I and N. Alw I (New England Biolabs® Inc, Beverly, USA). A detailed protocol for the application of N.Bpu 10I for the preparation of single-stranded DNA from supercoiled double-stranded plasmids containing an appropriate recognition site can be found on the website of Fermentas UAB under http://www.fermentas.com/ and this protocol is hereby incorporated herein by reference. The major drawback of the in vitro approaches to single-stranded DNA is again the contamination of preparations with double-stranded or at least partly double-stranded DNA molecules. Such contaminants have to be removed for many applications, and this is in particular true when the Gene II enzyme is used.

For the preparation of linear single-stranded DNA, various technologies have been developed familiar to a person skilled in the art. In many cases these approaches use a DNA polymerase based synthesis of single-stranded DNA from a DNA or RNA template. Any amplification method from linear template DNA or RNA yielding in an excess of single-stranded DNA over the template can be applied. Such approaches include the use of primed reactions driven by a DNA polymerase performed as an individual reaction or as a cyclic reaction to allow for a linear amplification of the product. For the preparation of high quality single-stranded DNA, it can be advisable to transcribe a DNA template first into RNA by means of a RNA polymerase. The template DNA can then be destroyed by means of a deoxyribonuclease before the RNA transcript is used as a template to synthesize single-stranded DNA thereof by means of a reverse transcriptase. By the use of two different forms of nucleic acids in the two independent reactions, the approach offers means for the removal of the templates by a deoxyribonuclease and a ribonuclease respectively. Though rather tedious to perform, this approach allows for the preparation of high quality linear single-stranded DNA. However, it does not apply for the preparation of circular single-stranded DNA, and it is dependent on appropriate promoter sites to drive a RNA polymerase and cleavage sites in the template for the termination of the transcription reaction.

In a particular case, a synthesis of single-stranded DNA can be achieved by the so-called asymmetric PCR reaction, in which the two primers are used at different concentrations. After the rate-limiting primer is exhausted, the reaction switches from the exponential amplification of double-stranded DNA to the linear amplification of the one strand primed by the primer used in excess over the rate-limiting primer. In an alternative approach lambda exonuclease is used to digest the one strand of double-stranded DNA having a 5′-phosphorylated end. Such a template can be prepared in PCR reactions in which only one out of two primers is phosphorylated at the 5′-end. The lambda exonuclease, also denoted as “Strandase™”, is commercially available from Novagen, Madison, USA, and the documentation on its “Strandase™ ssDNA Preparation Kit”, Cat. No. 69202, is hereby incorporated herein by reference. Similarly, the enzyme can also be obtained as lambda exonuclease from Epicentre, Madison, USA (Cat. Nos. LE035H and LE032K).

For a number of applications of single-stranded linear DNA, the single-stranded DNA is prepared by means of the PCR reaction in which one of the two primers is specifically tagged. While not limited to it, a biotin label is most frequently applied to separate the tagged strand as well as the second undesired strand from the template DNA. This approach is of value particularly when the strand of interest is supposed to be used as attached to a matrix or any kind of solid support. The thus immobilized single-stranded DNA can be directly purified on the support and used in detection assays depending on strand specific preparation and isolation of single-stranded DNA. One such application includes, but is not limited to, the detection and characterization of SNPs in genomic DNA in, for example, the so-called DASH SNP detection system. This approach is described in US Patent Application US2001046670, which is hereby incorporated herein by reference. Though this approach is of high value for certain systems and applications, its use is restricted to the preparation of linear single-stranded DNA, and does not allow the preparation and handling of circular single-stranded DNA.

Although powerful means for the preparation of single-stranded DNA have been developed over time and are commonly used in many applications, the preparation of single-stranded DNA still tends to be limited by the purity of the DNA and the contamination by double-stranded DNA, which most frequently is used as a source in single-stranded DNA preparations.

Current approaches for separating single-stranded DNA from double-stranded DNS are often based on chromatography procedures including, but not limited to, the separation by hydroxyapatite chromatography, benzoylated-naphthoylated-DEAE-cellulose (BNDC), methylated albumin on bentonite (MAB), or methylated albumin on Kieselgur (MAK). Many of those chromatography-based approaches are tedious to apply and limited by their low recovery rates for the single-stranded DNA. Therefore, there is a continuous need for new approaches for separating single-stranded DNA from double-stranded DNA.

### SUMMARY OF THE INVENTION

The present invention relates to a method for the preparation of single-stranded DNA, and more specifically provides a satisfying solution for the removal of double-stranded and partly double-stranded DNA molecules from single-stranded DNA preparations. In particular, the use of specific enzymatic activities under the specified conditions disclosed herein allow for a convenient and timesaving procedure which is of high value to many applications requiring or dependent on the use of single-stranded DNA.

The present invention provides a method for removing double-stranded DNA molecules from a mixture of double-stranded and single-stranded DNA molecules, comprising the steps of adding to the mixture a single-stranded-DNA binding substance which binds to single-stranded DNA molecules, and adding to the same mixture a double-strand-specific endonulease which specifically cleaves double-stranded DNA molecules. The addition of the single-stranded-DNA binding substance may precede that of the double-strand-specific endonulease or the addition of the two can be done simultaneously depending on the nature and relative affinity of the two to DNA. If the affinity of the single-stranded-DNA binding substance is much stronger to DNA than that of the double-strand-specific endonuclease, they can be added together, but the affinity of the two are comparable, it may be preferred to add the former first then the latter last.

The invention is used to remove entirely or partly double-stranded DNA from preparations of single-stranded DNA. The single-stranded DNA can be prepared by any approach known to a person skilled in the art, including, but not limited to, the use of in vivo approaches using a helper phage, or in vitro approaches using different enzymatic activities. As such, the invention relates to the preparation of single-stranded DNA from a linear template or from a circular template by any method established in the field known to a person skilled in the art.

More particularly, the single-stranded DNA can be a linear DNA molecule or a circular DNA molecule which is closed by a covalent bond, and it can be prepared from a linear DNA or RNA template or a circular DNA molecule derived from a plasmid or a phagemid. Circular DNA molecules can include any DNA molecule, whose ends are covalently bond to each other. Such circular DNA molecules can initially be of natural origin like plasmids or genetically modified like a phagemid. Furthermore, circular DNA molecules can be modified by the means of recombinant DNA technologies as described above or given in more details by J. Sambrook and D. W. Russell, ibid. Independent from the starting material used in the preparation of the single-stranded DNA, the invention provides a means for the purification or enrichment of the single-stranded DNA over double-stranded DNA in any given context.

In a preferable embodiment, the invention encompasses the use of an enzymatic system to specifically remove one strand from a double-stranded DNA template. The invention provides methods for the preparation of circular single-stranded DNA molecules from circular double-stranded DNA molecules, in which one strand is specific to a substrate that has an enzymatic activity showing preferential affinity for one DNA strand of interest compared to the other DNA strand. The enzymatic activity marks specifically one strand for destruction by a second and unrelated enzymatic activity. Due to the interrelated, however in their nature distinct, action of two enzymatic activities, a double-stranded circular DNA molecule can be converted into a circular single-stranded DNA molecule.

The present invention encompasses the use of a double-strand-specific endonuclease for the double-strand-specific digestion of double-stranded DNA. The double-strand-specific endonuclease digests specifically double-stranded DNA while leaving single-stranded DNA uncleaved, said the double-strand-specific endonuclease has preferential affinity for double-stranded DNA compared to single-stranded DNA. Thus any double-strand-specific endonuclease or any mixture having a double-strand-specific endonuclease activity can be applied to perform the invention, where double-stranded DNA is digested in the presence of entirely or partly single-stranded DNA.

The double-strand-specific endonuclease according to the invention may be a mixture of four-base-pair cutters. Such restriction endonucleases have a recognition site comprising four constitutive nucleotides within a double-stranded DNA molecule.

Many such enzymes are known to a person trained in the art and can be commercially obtained from different suppliers.

Preferably, the double-strand-specific endonuclease is the Duplex-Specific Nuclease (DSN) from crab hepatopancres, as described by D. A. Shagin et al. in Genome Res. Vol. 12, 2002, pages 1935 to 1942, which is hereby incorporated herein by reference. DSN is characterized for its double-strand specificity which allowed the authors to use the enzyme for the detection of SNPs in double-stranded DNA (D. A. Shagin et al., ibid), and as further described by the provider Evrogen (Moscow, Russia), whose product information on DSN is hereby incorporated herein by reference (http://www.evrogen.com/index.shtml). Thus, DSN can be viewed as a preferred enzyme for the enzymatic activity applied to perform the present invention.

In one embodiment, DSN can be used for its single enzymatic activity to remove double-stranded DNA from a mixture comprising single-stranded DNA, partly single-stranded or partly double-stranded DNA, and entirely double-stranded DNA.

More preferably, DSN can be applied together with a substance having single-stranded-DNA binding affinity. This substance should have preferential affinity for single-stranded DNA compared to double-stranded DNA. Due to its higher binding affinity to single-stranded DNA, the substance predominantly binds to single-stranded DNA in mixtures comprising single-stranded DNA, partly single-stranded or partly double-stranded DNA, and entirely double-stranded DNA. Thus, such a substance has the ability to protect single-stranded DNA against unspecific cleavage by the double-strand-specific endonuclease.

Preferably, the single-stranded-DNA binding substance may have higher or even much higher binding affinity for single-stranded DNA than compared to the double-strand-specific endonuclease used to perform the invention. In reaction mixtures comprising single-stranded DNA, partly single-stranded or partly double-stranded DNA, and entirely double-stranded DNA, such a single-stranded-DNA binding substance having higher binding affinity for single-stranded DNA compared to the double-strand-specific endonuclease applied to the same reaction titrates single-stranded DNA from complexes formed by the single-stranded DNA and double-strand-specific endonuclease. By titrating the single-stranded DNA from the complexes composed of the single-stranded DNA and double-strand-specific endonuclease, the single-stranded-DNA binding substance increases the concentration of free double-strand-specific endonuclease in the reaction mixture, thus increasing the turnover rate of the double-strand-specific endonuclease digesting double-stranded DNA. Therefore, the invention encompasses a method for using double-strand-specific endonucleases more efficiently by increasing the turnover rate of enzymatic reactions by the addition of a single-stranded-DNA binding substance.

The invention further encompasses a method in which the single-stranded-DNA binding substance is a protein which may be naturally occurring or modified to change its binding characteristics and which is isolated from an organism as expressed in vivo or in vitro using techniques of recombinant DNA. This protein may also be of synthetic origin. Such a protein may have affinity for any kind of single-stranded DNA or RNA without any sequence specificity, though it is within the scope of the present invention to use also proteins binding to single-stranded DNA in a sequence specific or enhanced manner.

In another preferable embodiment, the invention refers to the use of a single-stranded-DNA binding protein including, but not limited to, SSB from E. coli, products of phage T4 Gene 32, adenovirus DBP, an antibody directed against single-stranded DNA, calf thymus UP1, or any mixture thereof. However, the invention is not limited to the aforementioned single-stranded-DNA binding proteins, as genomic sequencing projects along with directed cDNA cloning approaches have revealed many single-stranded-DNA binding proteins which have been found to be essential for DNA replication and repair in vivo from bacteria to human. Thus, any of those proteins has the potential to be prepared and applied to perform the invention.

The invention also encompasses the further removal of linear single-stranded DNA from preparations of circular single-stranded DNA by an additional treatment of such a mixture comprising linear and circular single-stranded DNAs by a single-stranded DNA specific exonuclease. Here, any exonuclease having specificity for linear single-stranded DNA can be applied and such exonuclease should have a higher specificity for linear single-stranded DNA compared to circular single-stranded DNA. Such exonucleases include, but not limited to, ExoI, and ExoVII. By including such an exonuclease treatment into the purification step the invention allows for the distinction between linear and circular single-stranded DNA molecules. Thus, it is within the scope of the invention to provide a particular means for the specific purification of linear or circular double-stranded DNA and the removal of linear single-stranded DNA from preparations of circular double-stranded DNA.

Many applications and technologies in molecular biology and biotechnology require the strand-specific preparation of single-stranded DNA. Such applications include, but are not limited to, the preparation of template DNA for sequencing, template DNA for strand-specific DNA synthesis including synthesis of labeled probes, the introduction of point mutations, hybridization experiments like the preparation of testers and drivers for subtractive hybridizations or the detection and isolation of individual clones in a plurality of DNA or RNA molecules, the detection of SNPs, and the preparation of microarrays. Thus any such application of single-stranded DNA as prepared by the methods disclosed herein is within the scope of the invention, and the invention offers the necessary means to provide single-stranded DNA to be used in such an application.

As outlined in the above, the invention provides a new approach for a fast, effective, reliable, robust, and easy to perform method for the preparation and purification of single-stranded DNA. Thus, the invention is of great value for any kind of applications which depend on the use of single-stranded DNA, and in the future the invention will further contribute to the development of new technologies based on the use of single-stranded DNA, which until now could not be moved forward due to the limitations in currently available technologies for the preparation and purification of single-stranded DNA.

### BRIEF DESCRIPTION OF THE DRAWINGS

FIG. 1 is diagrams showing the digestion of single- and double-stranded DNAs using DSN under different conditions. Three different reaction conditions are presented as indicated: A, Digestion of dsDNA by DSN in the presence of ssDNA at 37° C., where ssDNA can form secondary structures and such secondary structures are cleaved by DSN. Thus, ssDNA was shown to become a substrate for DSN at low temperatures. B, Digestion of dsDNA by DSN in the presence of ssDNA at 65° C., where ssDNA can only form only a few secondary structures. Thus, DSN showed a preferential specificity for dsDNA at high temperatures, whereas ssDNA remained mainly undigested. C, Digestion of dsDNA by DSN in the presence of ssDNA and SSB, wherein SSB binds to ssDNA and disrupts secondary structures within ssDNA. Thus, DSN shows a high specificity for dsDNA at any reaction temperatures between 37° C. and 65° C., whereas ssDNA remains undigested.

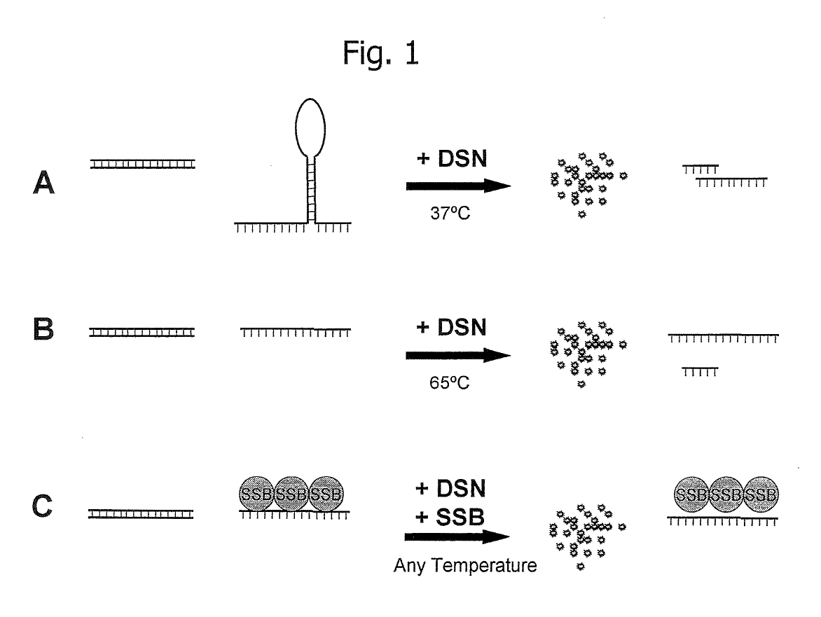

FIG. 2 shows the visualized results of the electrophoresis on samples which involves the use of DSN under different conditions. Linear single-stranded DNA was prepared from an mRNA sample denoted as “G2” as disclosed in the examples. Samples from different reaction conditions were applied to gel electrophoresis and DNA was visualized by SYBR Green II staining: Lane 1: Lambda/HindIII size marker, lane 2: G2-ssDNA incubated at 37° C., lane 3: G2-ssDNA incubated with DSN at 37° C., lane 4: G2-ssDNA incubated with DSN and SSB at 37° C., lane 5: G2-ssDNA incubated at 65° C., lane 6: G2-ssDNA incubated with DSN at 65° C., lane 7: G2-ssDNA incubated DSN and SSB at 65° C.

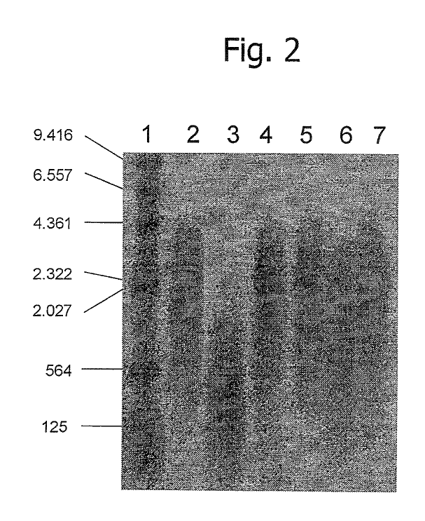

FIG. 3 shows diagrams explaining the principle for preparation of a circular single-stranded DNA. As presented in the figure, single-stranded DNA is prepared by means of different enzymatic actives in vitro, and additional treatments for the removal of byproducts are indicated.

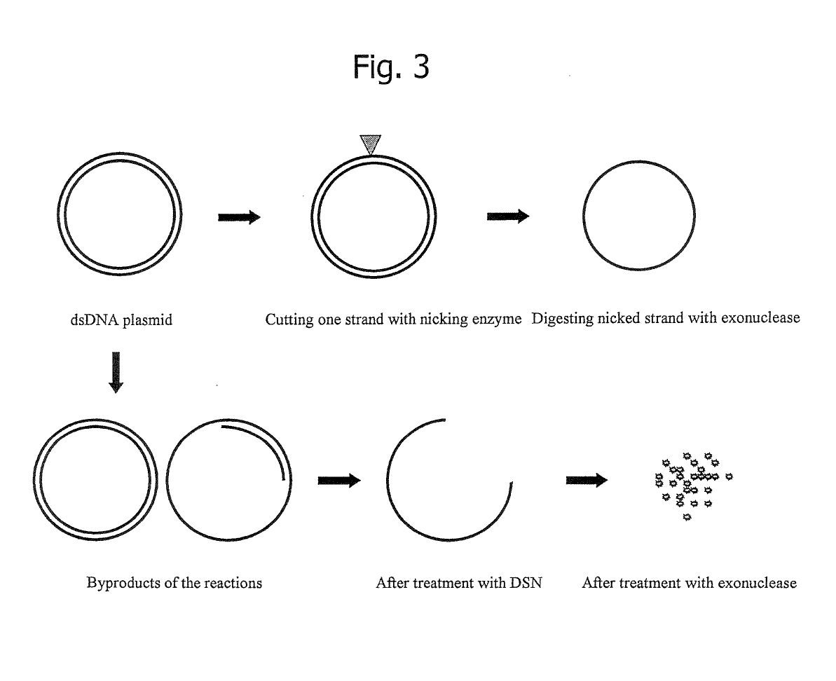

FIG. 4 shows reaction equations concerning reaction kinetics for DSN. DSN can interact with single-stranded and double-stranded DNAs as indicated in the figure, whereas it is competing with a single-stranded-DNA binding substance, here indicated as SSB, for binding to single-stranded DNA.

FIG. 5 shows the results of gel electrophoresis analysis imaged by autoradiography for digestion of single-stranded DNA by DSN in the presence and absence of the single-stranded-DNA binding proteins. A plurality of single-stranded DNA molecules of different sizes denoted as “G2” was treated with DSN under different conditions, and samples derived thereof were applied to gel electrophoresis and imaged by autoradiography: Lane 1: Lambda/HindIII size marker, lane 2: G2, lane 3: G2 plus DSN, lane 4: G2 plus T4 gene 32 product plus DSN, lane 5: G2 plus SSB plus DSN.

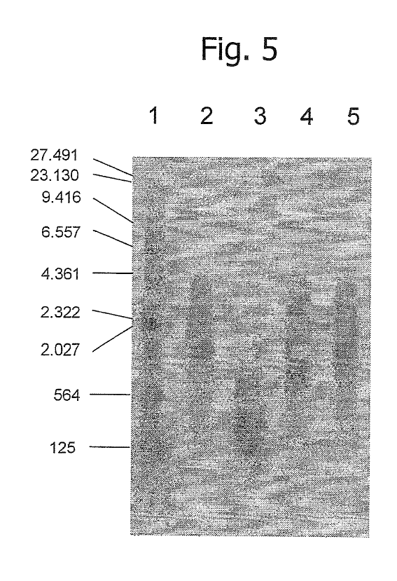

FIG. 6 shows the results of gel electrophoresis analysis for the preparation of single-stranded DNA from an individual vector by means of the present invention. Circular single-stranded DNA was prepared from a template of circular double-stranded DNA as disclosed in the examples. Samples from different steps of the preparation were applied to gel electrophoresis and DNA was visualized by SYBR Green II staining: Lane 1: Lambda StyI size marker, lane 2: vector pG2-1, lane 3: pG2-1plus GeneII, lane 4: pG2-1/GeneII plus ExoIII, lane 5: size marker as indicated for lane 1, lane 6: pG2-1/GeneII/ExoIII after purification, lane7: pG2-1/GeneII/ExoIII plus DSN, lane 8: pG2-1/GeneII/ExoIII plus T4 gene 32 product and DSN.

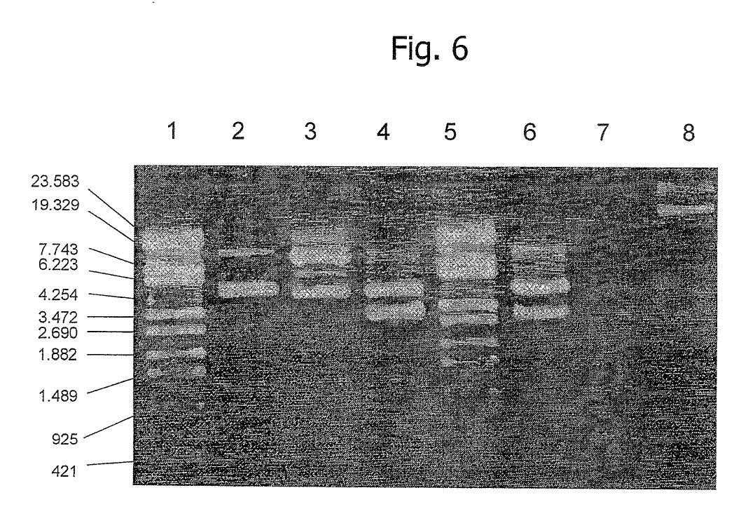

FIG. 7 shows the results of gel electrophoresis analysis for single-stranded DNAs prepared by means of the present invention. Single-stranded DNA samples as prepared by means of the invention and as shown in FIG. 4 were subjected to Proteinase K treatment to destroy DSN and complexes formed by a single-stranded-DNA binding substance and single-stranded DNA. Samples were applied to gel electrophoresis and DNA was visualized by SYBR Green II staining: Lane 1: Lambda StyI size marker, lane 2: pG2-1/GeneII/ExoIII/single-stranded-DNA binding substance/DSN DNA-protein complex before Proteinase K treatment, Lane 3: pG2-1/GeneII/ExoIII/single-stranded-DNA binding substance/DSN DNA-protein complex after Proteinase K treatment.

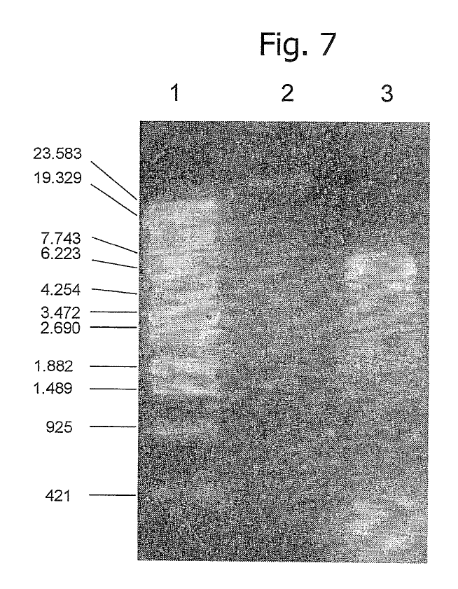

FIG. 8 shows the results of gel electrophoresis analysis for single-stranded DNA prepared by means of the invention. Circular single-stranded DNA samples as prepared by means of the invention and as shown in FIG. 5 were subjected to ExoI treatment to remove linear single-stranded DNA present in the preparations. Samples were applied to gel electrophoresis and DNA was visualized by SYBR Green II staining: Lane 1: Lambda StyI size marker, lane 2: pG2-1/GeneII/ExoIII/single-stranded-DNA binding substance/DSN/Proteinase K plus ExoI.

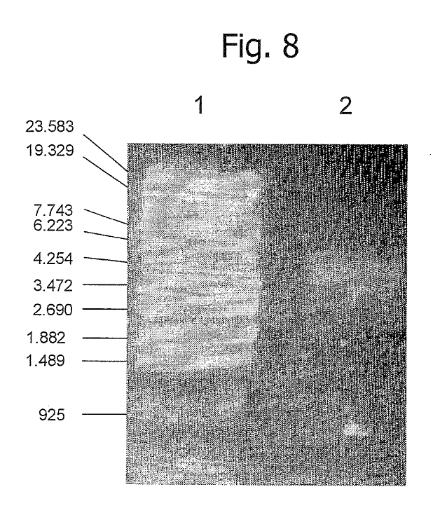

### DETAILED DESCRIPTION OF THE INVENTION

The present invention can be employed in a wide range of applications in gene discovery, genomic research, and manufacturing or services to produce recombinant DNA. The invention is also applicable to sciences in general related to life sciences and medical research. The methods disclosed herein are of high commercial value and contribute to many applications in the field of biotechnology. In particular the approach of the present invention will greatly contribute to academic and commercial research and development in the field in which single-stranded DNA is a requirement for the manipulation of nucleic acids.

Therefore, the invention encompasses methods for preparing single-stranded DNA from linear and circular double-stranded DNA templates. Double-stranded DNA means any nucleic acid molecules each of which is composed of two polymers formed by deoxyribonucleotides and in which the two polymers have substantially complementary sequences to each other allowing for their association to form a dimeric molecule. The two polymers are bound to one another by specific hydrogen bonds formed between matching base pairs within the deoxyribonucleotides. Any DNA molecule composed only of one polymer chain formed by two or more deoxyribonucleotides having no matching complementary DNA molecule to associate with is considered to be a single-stranded DNA molecule for the purpose of the invention, even if such a molecule may form secondary structures comprising double-stranded DNA portions. As used interchangeably herein, the terms “nucleic acid molecule(s)” and “polynucleotide(s)” include RNA or DNA regardless of single or double-stranded, coding or non-coding, complementary or not, and sense or anti-sense, and also include hybrid sequences thereof. In particular, it encompasses genomic DNA and complementary DNA which are transcribed or non-transcribed, spliced or not spliced, incompletely spliced or processed, independent from its origin, cloned from a biological material, or obtained by means of synthesis. More precisely, the expressions “DNA”, “RNA”, “nucleic acid”, and “sequence” encompass nucleic acid materials themselves and are thus not restricted to particular sequence information, vector, phagemid or any other specific nucleic acid molecule. The term “nucleic acid” is also used herein to encompass naturally occurring nucleic acids, artificially synthesized or prepared nucleic acids, any modified nucleic acids into which at least one or more modifications have been introduced by naturally occurring events or through approaches known to a person skilled in the art. The terms “purity”, “enriched”, “purification” or “enrichment” are used interchangeably herein and do not require absolute purity or enrichment of a product but rather are intended as relative definitions. The terms “specific”, “preferable”, or “preferential” are used interchangeably herein and do not require absolute specificity of an enzyme for its substrate or an activity, but rather they are intended to have relative definitions which include the possibility that an enzyme may have low or lower affinity to other compounds related or unrelated to its substrate. Similarly, the terms used to name an enzyme, an enzymatic activity, or single-stranded-DNA binding substance are used herein to describe the function or activity of such a component, but do not require the absolute purity of such a components. Thus any mixture containing such an enzyme, enzymatic activity, single-stranded-DNA binding substance or mixtures thereof with other components of the same, related or unrelated function are within the scope of the invention. The term “biological samples” includes any kind of material obtained from living organisms including microorganisms, animals, and plants, as well as any kind of infectious particles including viruses and prions, which depend on a host organism for their replication. As such “biological samples” include any kind material obtained from a patient, animal, plant or infectious particle for the purpose of research, development, diagnostics or therapy. Thus, the invention is not limited to the use of any particular nucleic acid molecules or their origin, but the invention provides general means to be applied to and used for the work on and the manipulation of any given nucleic acid. Any such nucleic acid molecules as applied to perform the invention can be obtained or prepared by any method known to a person skilled in the art.

In a preferred embodiment, the invention is used to remove entirely or partly double-stranded DNA molecules from a preparation of single-stranded DNA. The single-stranded DNA can be prepared by any approach known to a person skilled in the art, including, but not limited to, the use of in vivo approaches using helper phages or in vitro approaches using different enzymatic activities.

As such the invention relates to the preparation of single-stranded DNA from a linear template or from a circular template by any method established in the field as known to a person skilled in the art.

More particularly, the single-stranded DNA can be a linear DNA molecule or a circular DNA molecule closed by a covalent bond, and it can be prepared from a linear DNA or RNA template or, a circular DNA molecule, or obtained from plasmids or phagemids. Independent from the starting material used in the preparation of the single-stranded DNA, the invention provides means for the purification or enrichment of the single-stranded DNA over double-stranded DNA in any given context.

Any approach known to a person skilled in the art that allows for the preparation of single-stranded DNA can be used to perform the invention. Thus, the invention does not depend on a particular approach for the preparation of single-stranded DNA, but can be applied to any known approach. In one preferred embodiment, the invention makes use of linear single-stranded DNA. Template DNA for the preparation of linear single-stranded DNA can be circular or linear RNA or DNA, already single-stranded or double-stranded by nature, and can be obtained, prepared or modified by any method known to a person skilled in the art. For the preparation of linear single-stranded DNA various technologies have been developed as familiar to a person skilled in the art including, but not limited to, the ones named below. In many cases, these approaches use a DNA polymerase based synthesis of single-stranded DNA from a DNA or RNA template. Any amplification method using a linear template DNA or RNA yielding in an excess of single-stranded DNA over the template can be applied for the invention. Such approaches include, but are not limited to, the use of primed reactions driven by a DNA polymerase performed as an individual reaction or as a cyclic reaction. Such DNA polymerases include, but are not limited to, the Klenow fragment of DNA polymerase I, T4 and T7 DNA polymerases, DNA polymerase I, Taq polymerase, Tfl DNA polymerase, Tth DNA polymerase, Tli DNA polymerase, or any other DNA polymerase known in the field. High quality single-stranded DNA can further be prepared by transcribing a DNA template first into RNA by means of a RNA polymerase including, but not limited to, T4-, T7-, or SP6 RNA polymerase, where the transcription reaction can be terminated by linearization of the template. The template DNA can then be destroyed by means of a deoxyribonuclease before the RNA transcript is used as a template for the synthesis of single-stranded DNA by means of a reverse transcriptase. Such reverse transcriptases include, but are not limited to, AMV reverse transcriptase, M-MLV reverse transcriptase or M-MLV reverse transcriptase RNase H minus. By the use of two different forms of nucleic acids in the two independent reactions, the approach offers a means for the removal of the templates by a deoxyribonuclease and a ribonuclease respectively.

In a particular case, the synthesis of single-stranded DNA can further be achieved by the so-called asymmetric PCR reaction, in which the two primers are used at different concentrations. After the rate-limiting primer is exhausted, the reaction switches from the exponential amplification of double-stranded DNA to the linear amplification of the one strand primed by the primer used in excess over the rate-limiting primer. In an alternative approach lambda exonuclease is used to digest the one strand of double-stranded DNA having a 5′-phosphorylated end. Such a template can be prepared in PCR reactions in which only one out of two primers is phosphorylated at the 5′-end. The enzyme, lambda exonuclease also denoted as “Strandase ”, is commercially available from Novagen, Madison, USA, and the documentation on its “Strandase™ ssDNA Preparation Kit”, Cat. No. 69202, is hereby incorporated herein by reference. Similarly, the enzyme can also be obtained as lambda exonuclease from Epicentre, Madison, USA (Cat. No. LE035H and LE032K). In a related approach, the single-stranded DNA is prepared by means of the PCR reaction in which one of the two primers is specifically tagged including, but not limited to, a biotin label, digoxigenin, or an amino group. Modified oligonucleotides can be obtained from most suppliers providing DNA synthesis services and are easily available to perform the invention. The tag can be used to separate the tagged strand from the template DNA as well as the second undesired strand. This approach is particularly of value if the strand of interest is supposed to be used as attached to a matrix or any kind of solid support. The thus immobilized single-stranded DNA can be directly purified on the support and used in detection assays depending on strand specific preparation and isolation of single-stranded DNA.

However, the invention in not limited to the preparation of linear single-stranded DNA, as the invention relates also to the preparation of circular single-stranded DNA. Template DNA for the preparation of circular single-stranded DNA can be circular or linear RNA or DNA, already single-stranded or double-stranded by nature, derived from plasmids, phagemids, self-ligated molecules, and can be obtained, prepared or modified by any method known to a person skilled in the art. Circular single-stranded DNA can be prepared by any method known to a person skilled in the art. This includes, but is not limited to, approaches that make use of a helper phage for in vivo synthesis of single-stranded circular DNA. Standard approaches for this kind of preparation of single-stranded DNA commonly use, but are not limited to, phagemids, which have the cis-acting regulatory sequences for the initiation and termination of DNA synthesis from the bacteriophage M13 genome. Thus, such phagemids allow for the in vivo preparation of single-stranded DNA when the host bacteria are infected by a helper wild type or mutant filamentous bacteriophage carrying replication-defective intergenic regions. Such helper phages include, but are not limited to, the interference-resistance helper phages R408 (available from Stratagene, La Jolla, USA, Cat. No. 200252, or Promega, Madison, USA, Cat. No. P2291 and P2341), VCSM13(Stratagene, La Jolla, USA, Cat. No. 200251), or M13KO7 (New England Biolabs®, Beverly, USA, Cat No. N0315). After infection of a bacterial culture of bacteria carrying an appropriate phagemid with an interference-resistant helper phage, the gene II product encoded by the helper phage introduces a strand-specific nick into the intergenic region of the phagemids initiating a rolling-circle like replication of one strand. Thereafter, single-stranded copies of the phagemid DNA are packed into the progeny bacteriophage particles and extruded into a medium from which the single-stranded DNA can be isolated or purified by standard methods as known to a person skilled in the art and described by J. Sambrook and D. W. Russell, ibid.

Moreover, the invention relates to the in vitro preparation of circular single-stranded DNA by means of different enzymatic activities. Template DNA for the preparation of circular single-stranded DNA can be circular or linear RNA or DNA, already single-stranded or double-stranded by nature, and can be obtained, prepared or modified by any method known to a person skilled in the art. As an alternative method to the in vivo preparation of single-stranded DNA, in vitro approaches have been developed, which make use of combinations of two different enzymatic activities, as familiar to a person skilled in the art. Most commonly, a combination of the replication initiator protein Gene II of the bacteriophage f1 and ExoIII is used in such systems. The Gene II enzyme will act as a site-specific endonuclease that recognizes the f1 ori in a phagemid vectors, and cleaves the viral strand. ExoIII will attack the free 3′-end of the nicked strand and digest it until the other strand is released as single-stranded circular DNA. Such a system can be commercially obtained, e.g., as part of the so-called GeneTrapper® cDNA Positive Selection System from Gibco BRL/Life Technologies (CAT. NO. 10356-020, nowadays part of Invitrogen Corporation, Carlsbad, USA), the Instruction Manual of which is hereby incorporated herein by reference. Any other strand-specific nicking enzymes can be used as well to perform the invention if such an enzyme cleaves only one DNA strand within its recognition sequence in a double-stranded DNA substrate. Such enzymes include, but are not limited to, the commercially available nucleases N.Bpu 10I (FERMENTAS UAB, Vilnius, Lithuania), N.Bbv C IA, N.Bst NB I and N. Alw I (New England Biolabs Inc, Beverly, USA). A detailed protocol for the application of N.Bpu 10I for the preparation of single-stranded DNA from supercoiled double-stranded plasmids containing the appropriate recognition site can be found on the website of Fermentas UAB under http://www.fermentas.com/ and is hereby incorporated herein by reference. Similarly, the invention is not limited to the use of ExoIII as the enzyme can be replaced by any other exonuclease which can digest one DNA strand in double-stranded DNA.

Any single-stranded DNA independently from its method of preparation has to be purified to a certain extend to allow for its use in any applications of interest. Means for enrichment or purification may vary on the experimental needs. Many approaches for the purification of single-stranded DNA are well known to a person skilled in the art. Such purification steps can include, but are not limited to, the use of ribonucleases to remove RNA, proteases, including, but not limited to, Proteinase K, to remove remaining proteins, the extraction with phenol to remove proteins, gel filtration on an appropriate matrix or through an appropriate membrane, gel electrophoresis, the application of chromatographic approaches including, but not limited to, a matrix or substance having affinity for single-stranded DNA, the use of a single-stranded-DNA binding substance, the use of commercially available kits, and the precipitation of the DNA by ethanol or propanol for concentrating the sample. Furthermore, there are approaches to remove specific byproducts by an enzymatic activity as outlined above, or chromatographic procedures including, but not limited to, the separation on hydroxyapatite chromatography, benzoylated-naphthoylated-DEAE-cellulose (BNDC), methylated albumin on bentonite (MAB), or methylated albumin on Kieselgur (MAK), which may be included or intentionally excluded while performing the invention.

The invention encompasses the use of a double-strand-specific endonuclease for the double-strand-specific digestion of double-stranded DNA. The double-strand-specific endonuclease digests specifically double-stranded DNA while leaving single-stranded DNA uncleaved, and the double-strand-specific endonuclease has preferential affinity for double-stranded DNA compared to single-stranded DNA. Thus, any double-strand-specific endonuclease can be used to perform the invention, so as to digest double-stranded DNA in the presence of entirely or partly single-stranded DNA. Under the conditions disclosed herein, the double-strand-specific endonuclease digests remaining double-stranded DNA, and removes the double-stranded part of DNA molecules which are in part composed of single-stranded and double-stranded DNA. Such partly double-stranded DNA molecules are derived from double-stranded DNA when, for example, the digestion of the nicked DNA strain by the endonuclease remains incomplete. As a result, the double-strand-specific endonuclease releases linear single-stranded DNA from substrates including partly single-stranded and partly double-stranded DNA. Such single-stranded DNA can be further digested by means of a single-stranded DNA specific exonuclease as outlined below.

Similarly, the double-strand-specific endonuclease can remove double-stranded or partly double-stranded DNA from any preparation of single-stranded DNA, independent from the method used for its preparation, nature, origin, whether linear or circular. Thus, the invention relates to a general approach for the removal of double-stranded DNA from any preparation of partially and entirely single-stranded DNA.

In a preferable embodiment, the double-strand-specific endonuclease is a mixture of four-base-pair cutters which are restriction endonucleases having a recognition site comprising four constitutive nucleotides within a double-stranded DNA molecule. Many such enzymes are known to a person skilled in the art, and can be commercially obtained from different suppliers including, but not limited to, FERMENTAS UAB (Vilnius, Lithuania), New England Biolabs Inc. (Beverly, USA), Promega (Madison, USA), Takara (Tokyo, Japan), Roche (Mannheim, Germany), and Amersham Biosciences (Cardiff, United Kingdom). Such restriction endonucleases which cut only double-stranded DNA but do not cut single-stranded DNA include, but are not limited to, the four-base-pair cutters, HapII, HypCH4IV, AciI. HhaI, MspI, AluI, BstUI, DpnII, HaeIII, MboI, NlaIII, RsaI, Sau3AI, Taq alpha I, TspRI, BsrI, MnlI, BfaI, MaeI, PleI, MseI, HinPlI, and Tsp 509I, out of which candidates can be selected for the preparation of any given mixture thereof or in combination with any other enzymes. Other suitable restriction endonucleases which are apparent to those skilled in this field can be applied as well to perform the invention. Thus, the invention is not limited to the use of a particular enzyme or a particular mixture of enzymes. On average, a four-base-pair cutter which is a restriction endonuclease having a recognition site comprising four constitutive nucleotides within a double-stranded DNA molecule cleaves a double-stranded DNA molecule of random sequence about every few hundred base pairs. Therefore, any mixture of such four-base-pair cutters would allow for the digestion of double-stranded DNA in the presence of single-stranded DNA, if double-stranded DNA molecules are fragmentized into oligonucleotides of a few nucleotides in length. Short oligonucleotides can easily be removed from larger DNA fragments by standard methods known to a person skilled in the art. Such approaches include, but are not limited to, methods established for the removal of primers from PCR reactions as commercially available from Promega (Madison, USA), Qiagen (Hilden, Germany), and Invitrogen (Carlsbad, USA).

In a more preferable embodiment, the double-strand-specific endonuclease is DSN from crab hepatopancres, as described by D. A. Shagin et al., ibid, which publication is incorporated herein by reference, and as further described by the provider Evrogen (Cat# EA001, Moscow, Russia), whose product information on DSN is incorporated herein by reference (http://www evrogen.com/index.shtml). DSN is characterized for its preferential specificity for double-stranded DNA and has a higher specificity for double-stranded DNA compared to single-stranded DNA. However, its specificity for double-stranded DNA is dependent on the reaction temperature as single-stranded DNA can form secondary structures which also include stretches of double-stranded DNA. Thus, the use of DSN in the presence of single-stranded DNA is limited to short single-stranded DNA molecules which do not form stable secondary structures or requires high reaction temperatures at which most secondary structures are disrupted. Similarly, DSN can remove the DNA from double-stranded hybrids composed of one RNA and one DNA strand. Furthermore it is within the scope of the invention to use DSN on any nucleic acid molecule partly or entire composed of modified nucleotides. Such modifications as known to a person skilled in the art may or may not interfere with the enzymatic activity of the enzyme. Therefore, it can be envisioned to use such modified nucleotides to protect certain areas within a nucleic acid molecule against digestion by DSN. Thus, DSN can be viewed as a preferred enzyme for the enzymatic activity used in performing the invention, because DSN has the ability to digest double-stranded DNA in the presence of single-stranded DNA.

Optionally, DSN can be applied in an appropriate buffer system at any temperature from 4° C. to 65° C. Under more preferable conditions, the reaction can be performed at 37° C. or 50° C. Under even more preferable conditions, the reaction can be performed at about 65° C., at which temperature most of the secondary structures in single-stranded DNA molecules dissociate. Furthermore, single-stranded DNA as subjected to the DSN treatment can be incubated at 65° C. before performing the enzymatic reaction. The dissociation of secondary structures is important for the use of any double-strand-specific endonuclease including, but not limited to, DSN, as secondary structures can include stretches of double-stranded DNA formed by complementary sequences within the single-stranded DNA molecule. Any such stretches of double-stranded DNA within a single-stranded DNA molecule can be recognized by the double-strand-specific endonuclease, leading to the destruction of the molecule. Similarly, stretches of single-stranded DNA in two different single-stranded DNA molecules having sequences complementary to each other can lead to the formation of hybrid molecules with stretches of double-stranded DNA. In such a case, it is desirable to dissociate such double-strand structures before the treatment using a double-strand-specific endonuclease.

In one embodiment, DSN can be used for a single enzymatic activity to remove double-stranded DNA from mixture comprising single-stranded DNA, partly single-stranded as well as partly double-stranded DNA, and entirely double-stranded DNA.

In another embodiment, DSN can be used for its enzymatic activity in combination with four-base-pair cutters which are restriction endonucleases having a recognition site comprising four constitutive nucleotides within a double-stranded DNA molecule, in order to remove any double-stranded DNAs from a mixture comprising single-stranded DNA, partly single-stranded as well as partly double-stranded DNA, and entirely double-stranded DNA.

Preferably, DSN is used together with a substance having single-stranded-DNA binding affinity which has preferential affinity for single-stranded DNA compared to double-stranded DNA. Due to its higher binding affinity to single-stranded DNA, such substance predominantly binds to single-stranded DNA in mixtures comprising single-stranded DNA, partly single-stranded as well as partly double-stranded DNA, and entirely double-stranded DNA. Thus, such substance is capable of protecting single-stranded DNA against unspecific cleavage by the double-strand-specific endonuclease.

Preferably, the single-stranded-DNA binding substance can disrupt secondary structures in single-stranded DNA molecules. The dissociation of secondary structures is important for the application of any double-strand-specific endonuclease including, but not limited to, DSN, as secondary structures can include stretches of double-stranded DNA formed by complementary sequences within the single-stranded DNA. Any stretch of double-stranded DNA within a single-stranded DNA molecule can be recognized by the double-strand-specific endonuclease, leading to the destruction of the molecule. Thus, the disruption of secondary structures to obtain a linear structure by means of a single-stranded-DNA binding substance can protect single-stranded DNA against unspecific cleavage by the double-strand-specific endonuclease.

Further preferably, the single-stranded-DNA binding substance should have higher binding affinity for single-stranded DNA than the double-strand-specific endonuclease used to perform the invention. Even further preferably, the single-stranded-DNA binding substance has much higher binding affinity for single-stranded DNA than the double-strand-specific endonuclease used to perform the invention. In reaction mixtures comprising single-stranded DNA, partly single-stranded as well as partly double-stranded DNA, and entirely double-stranded DNA, a single-stranded-DNA binding substance having higher binding affinity for single-stranded DNA compared to the double-strand-specific endonuclease applied to the same reaction titrates single-stranded DNA from complexes formed by single-stranded DNA and a double-strand-specific endonuclease. By titrating single-stranded DNA from complexes of single-stranded DNA and double-strand-specific endonuclease, the single-stranded-DNA binding substance increases the concentration of free double-strand-specific endonuclease molecules in the reaction mixture, thus increasing the turnover rate of the double-strand-specific endonuclease digesting double-stranded DNA. Therefore, the invention encompasses a method for improving the enzymatic activity of double-strand-specific endonucleases by the addition of a single-stranded-DNA binding substance because it increases the concentration of free double-strand-specific endonuclease molecules in the reaction mixture, thus allowing for an enhanced turnover rate of the enzyme in such an enzymatic reaction. This mechanism is distinct from previously reported applications of single-stranded-DNA binding substances in enzymatic reactions in which the single-stranded-DNA binding substance acts by stabilizing single-stranded DNA in complexes formed between a single-stranded DNA molecule and a single-stranded-DNA binding substance.

Any substance having the ability to bind to or to associate with single-stranded DNA can be applied to perform the invention, including, but not limited to, the use of chemical compounds, a matrix, a solid support modified to change its binding specificity, or a protein. Preferentially, such a single-stranded-DNA binding substance should have no sequence specificity to allow for a general application of the invention. However, it can be envisioned that the use of particular sequence specific or sequence enhanced single-stranded-DNA binding substances can be applied as well to perform the invention if such a substance binds to a sequence of interest used to perform the invention or if the recognition motif of such a substance is so short that it frequently occurs within any given single-stranded DNA. Such a single-stranded-DNA binding substance includes, but is not limited to, the potent anti-tumor drug Actinomycin D (AMD), which is shown to have a preference for binding to specific tri-nucleotide motives in single-stranded DNA (R. M. Wadkins et al., J. Mol. Biol., Vol. 262, 1996, pages 53 to 68, which is hereby incorporated herein by reference).

The invention further encompasses a method in which the single-stranded-DNA binding substance is a protein, naturally occurring or modified to change its binding characteristics, isolated from an organism, expressed in vivo or in vitro using techniques of recombinant DNA, or of synthetic origin. Such a protein may have affinity for any kind of single-stranded DNA or RNA without any sequence specificity, though it is within the scope of the invention to use also proteins binding to single-stranded DNA in a sequence specific or enhanced manner as outlined above.

In a more preferable embodiment, the invention refers to the use of single-stranded-DNA binding protein including, but not limited to, SSB from E. coli, the product of the phage T4 Gene 32, the adenovirus DBP, an antibody directed against single-stranded DNA, calf thymus UP1, or any mixture thereof SSB from E. coli is commercially available from various providers including, but not limited to, Stratagene, La Jolla, USA (Cat. No. 600201), Promega, Madison, USA (Cat. No. M3011), Amersham Biosciences, Cardiff, United Kingdom (Cat. No. E70032Y), and Epicentre, Madison, USA (Cat. No. SSB02200). It is commonly used in reactions depending on single-stranded DNA like sequencing reactions in which SSB maintains the denaturation of secondary structures, which could otherwise inhibit chain elongation by DNA polymerases. Similarly, it has been found to improve digestion by restriction endonuclease, enhance the specificity and yield of PCR reactions, improve site-directed mutagenesis in conjunction with the recA protein, and improve the action of DNA polymerases in DNA replication. Other single-stranded-DNA binding proteins can be obtained in the public domain, including, but not limited to, the product of the phage T4 Gene 32. The product of the phage T4 Gene 32 is commercially available from various providers including, but not limited to, Nippon Gene, Tokyo, Japan (Cat. No. 312-03251), USB, Cleveland, USA (Cat. No. 74029Y) and Amersham Biosciences, Cardiff, United Kingdom (Cat. No 25003911). In addition, autoantibodies against single-stranded DNA are found frequently in patients with nonrheumatic diseases including chronic active hepatitis and infectious mononucleosis. Such autoantibody can be purified by affinity-purification on a DNA matrix or obtained by immunization of an animal. Such antibodies can further be obtained in the public domain for diagnostic purpose e.g. in enzyme immunoassays. A human antibody against single-stranded DNA is commercially available from various providers including, but not limited to, Immunovision, Springdale, USA (Code HSS-0100).

However, the invention is not limited to the aforementioned single-stranded-DNA binding proteins, as genomic sequencing projects along with directed cDNA cloning approaches have revealed many single-stranded-DNA binding proteins which have been found essential for DNA replication and repair in vivo from bacteria to human. Thus, any of those proteins is within the scope of the invention and has the potential to be prepared and applied to perform the invention as disclosed herein for other single-stranded-DNA binding proteins.

Single-stranded-DNA binding proteins including, but not limited to, the ones named above can be used under the same reaction conditions as the double-strand-specific endonuclease. Preferable reactions can be carried out at a temperature of 37° C. More preferable reactions can be carried out at a temperature of 50° C. Even more preferable reactions can be carried out at a temperature of 65° C. DSN and SSB are thermostable proteins which are functional at temperatures of up to 65° C. Thus, the invention is not limited to the use of a double-strand-specific endonuclease and a single-stranded-DNA binding substance at any given temperature, but any appropriate reaction temperature can be applied depending on experimental needs.

In an even more particular embodiment, the invention also encompasses the further removal of linear single-stranded DNA from preparations of circular single-stranded DNA by an additional treatment of such a mixture comprising of linear and circular single-stranded DNA by means of a single-stranded DNA specific exonuclease. Any exonuclease having specificity for linear single-stranded DNA can be applied to perform the invention, if the exonuclease has a higher specificity for linear single-stranded DNA compared to circular single-stranded DNA. Such enzymes include, but are not limited to, the exonucleases ExoI, and ExoVII. Reaction conditions for those enzymes are well known to a person skilled in the art and are further described by J. Sambrook and D. W. Russell, ibid.

The preparation of single-stranded DNA and its quality can be analyzed by agarose gel electrophoresis as described by J. Sambrook and D. W. Russell, ibid. In an agarose gel of a given concentration, nicked double-stranded DNA migrates as open circular DNA which shows a slower migration pattern than supercoiled DNA which can be the substrate of the nicking enzyme. Similarly, single-stranded circular DNA can be distinguished from supercoiled DNA and open circular DNA by its faster migration pattern moving ahead of supercoiled and open circular DNA. Thus, agarose gel electrophoresis is an appropriate tool to distinguish between the different stages of the single-stranded DNA preparation, and allows monitoring the quality of the single-stranded DNA. It can further be applied to monitoring the purification of single-stranded DNA from mixtures composed of heterogeneous DNA molecules.

Single-stranded DNA can also be quantified in calorimetric or fluorescence assays by reagents specifically or preferentially binding to single-stranded DNA. One such reagent includes, but is not limited to, OliGreen®, a sensitive fluorescent nucleic acid stain, commercially available from Molecular Probes, Eugene, USA (Cat. No. O-7582and O-11492). In contrast to the commonly used measuring of DNA concentrations by determination their absorbance at 260 nm, OliGreen® does not interfere with contaminating nucleotides and shows a much higher sensitivity. Therefore, it applies for good reason to the quantification of single-stranded DNA obtained by means of the invention, as nucleotides and short oligonucleotides of up to six bases are not detected. Thus OliGreen® does not interfere with free nucleotides or short oligonucleotides derived from the digestion of double-stranded DNA by means of a double-strand-specific endonuclease or that of linear single-stranded DNA by means of a linear single-stranded DNA specific exonuclease. However, OliGreen® does exhibit fluorescence enhancement by interaction of double-stranded DNA or RNA.

Methods for the strand-specific preparation of single-stranded DNA are needed for many technologies and applications in the field. Thus, the invention encompasses means for the preparation of high-quality single-stranded DNA and its use in applications known to a person skilled in the art.

DNA sequences can be determined by different chemical or enzymatic reactions by technologies known to a person skilled in the art. All those approaches make use of single-stranded DNA templates, which are the substrate for nucleotide specific chemical reactions, or enzymatic reactions, in which the single-stranded DNA functions as a template for the synthesis of the complementary strand. J. Sambrook and D. W. Russell (ibid, hereby incorporated herein by reference) described standard approaches for DNA sequencing as known to a person skilled in the art.

Single-stranded DNA is commonly used as a template for the introduction of point mutations. Preferentially, DNA from a plasmid or phagemid is converted into circular single-stranded DNA, and an oligonucleotide harboring the desired mutation is hybridized against the circular single-stranded DNA and used as a primer to synthesis the second strand by technologies known to a person skilled in the art or described by J. Sambrook and D. W. Russell, ibid.

Detection Methods

Single-stranded DNA is further used for the detection and isolation of individual clones in a plurality of DNA or RNA molecules including, but not limited to, the use in the so-called GeneTrapper® cDNA Positive Selection System from Gibco BRL/Life Technologies (CAT. NO. 10356-020, nowadays part of Invitrogen Corporation, Carlsbad, USA), the Instruction Manual of which is hereby incorporated herein by reference.

Single-stranded DNA is further used for the preparation of modified or labeled DNA probes that are used in hybridization experiments or other approaches known to a person skilled in the art. Such applications include, but are not limited to, the incorporation of uracil, use of radioactive nucleotides, or non-radioactive labels including, but not limited to, biotin and digoxigenin.

Single-stranded DNA is further used in assays for the detection of SNPs in genomic or transcripted DNA, which depend on the strand specific preparation of one strand for analysis. Such approaches include, but are not limited to, the so-called DASH SNP detection system, US Patent Application US2001046670, ibid.

Directed Cleavage

Single-stranded DNA is further used to create specific cleavage sites for endonucleases. In one such an application, an oligonucleotide having a defined sequence complementary to the desired site for cleavage is hybridized to a single-stranded DNA template. In the region, where the oligonucleotide binds to the single-stranded DNA, a stretch of double-stranded DNA which comprises a recognition site for a restriction endonuclease is formed. Thus, a specific region within the single-stranded DNA can be selected for cleavage, even if the double-stranded DNA template initially used for the preparation of the single-stranded DNA template contained many recognition sites for the restriction endonuclease of choice. As the restriction endonucleases depend on the presence of defined recognition sites within the target DNA, it may be desirable to allow for sequence-independent cleavage of the DNA template. In this embodiment of the invention, an oligonucleotide having the desired sequence as selected for the point of cleavage is hybridized to a single-stranded DNA template, and the stretch of double-stranded DNA comprising the oligonucleotide and the template DNA is cleaved by means of a double-strand-specific endonuclease. In a preferred embodiment of the invention, the double-strand-specific endonuclease is DSN. In a more preferable embodiment of the invention, the double-strand-specific endonuclease is used in the presence of a single-stranded-DNA binding substance. In an even further preferable embodiment of the invention, the double-strand-specific endonuclease is DSN, which is used in the presence of a single-stranded-DNA binding substance. Thus, the invention provides a means for the site-specific cleavage of a single-stranded DNA template by means of a double-strand-specific endonuclease.

Subtractive PCR

In this embodiment, the invention allows further for the performance of subtractive PCR reactions on a plurality of nucleic acid molecules. In this substractive PCR, one or more oligonucleotides are designed to target a subset of nucleic acids within a larger pool of nucleic acid molecules. The target nucleic acid molecules in the subset are collectively called a tester, and the one or more oligonucleotides in excess amounts are collectively called a driver. The oligonucleotides may have a sequence selected from any genomic DNA or DNA or RNA as of transcripted regions, and they may comprise regions derived from introns or exons. Also, sequences from unrelated organisms or sources could be included here, as it can be desirable for example to remove transcripts derived from a parasite when cloning the genomic information from the host. Thus, the oligonucleotide sequences are selected based on the experimental needs and the target nucleic acid molecules to be removed from the larger pool.

To achieve the necessary specificity, certain bioinformatics tools as known to a person skilled in the art can be applied to choose representative motifs within the target sequences. Oligonucleotides comprising the selected sequences can be obtained by chemical synthesis, as routinely offered by many suppliers on the market.

To perform this embodiment of the invention the driver as given in the form of specific oligonucleotides is incubated with the tester as given in the form of single-stranded DNA to allow for the hybridization of complementary regions. The oligonucleotides bind within the target sequence of the PCR reaction as flanked by the primer sites used to perform the PCR reactions, and a specific oligonucleotide from the driver binds to a portion within the tester sequences, which portion lies within the amplified region as marked by the flanking primer sites. Thus, after the binding of the oligonucleotides to their target sequences, templates chosen for destruction are cleaved by means of a double-strand-specific endonuclease, and the double-strand-specific endonuclease can only cleave double-stranded DNA regions comprising an oligonucleotide hybridized to a sequence within the tester. In contrast, sequences in the tester for which no matching oligonucleotide is present in the driver remain as entirely single-stranded DNA and thus remain unharmed from the treatment with the double-strand-specific endonuclease. After having performed the reaction step with the double-strand-specific endonuclease, the double-strand-specific endonuclease is removed from the DNA template, and the template is subjected to amplification by standard means known to a person skilled in the art. During the amplification step, only templates comprising both primer sites are amplified, whereas tester sequences that have been the targets of the double-strand-specific endonuclease are no longer available for amplification.

In a preferred embodiment of the invention, the double-strand-specific endonuclease is DSN. In a more preferable embodiment of the invention, the double-strand-specific endonuclease is used in the presence of a single-stranded-DNA binding substance. In an even more preferable embodiment of the invention, the double-strand-specific endonuclease is DSN, which is used in the presence of a single-stranded-DNA binding substance. Thus, the invention provides means for the site-specific cleavage of a single-stranded DNA template by means of a double-strand-specific endonuclease. Furthermore, the invention provides means for the targeted cleavage of a subset of nucleic acid molecules within a plurality of nucleic acid molecules, in which nucleic acid molecules of free choosing can be targeted and cleaved in a sequence independent manner. As cleaved by means of the invention, those target molecules can no longer be used as templates for the PCR reaction, thus allowing for the application of the invention in subtractive PCR reactions.

Microarrays

Single-stranded DNA is further used for the preparation of cDNA microarrays in which the use of single-stranded DNA is essential for the detection of specific target sequences. Microarrays can be prepared by various technologies known to a person skilled in the art.

In yet another embodiment the probes as attached to the microarray could comprise single-labeled or double-labeled single-stranded oligonucleotides. In such an application each oligonucleotide as present on the array would give raise to a signal in a detection system. After specific binding of complementary nucleic acid molecules as presented by a sample to such a labeled oligonucleotide on the array, a stretch of double-stranded DNA would be formed on the array. These stretches of double-stranded DNA can be cleaved by means of a double-strand-specific endonuclease to destroy the labeled oligonucleotide, and thus to destroy the signal on the array. In a preferred embodiment of the invention, the double-strand-specific endonuclease is DSN. In a more preferable embodiment of the invention, the double-strand-specific endonuclease is used in the presence of a single-stranded-DNA binding substance. In an even more preferable embodiment of the invention, the double-strand-specific endonuclease is DSN, which is used in the presence of a single-stranded-DNA binding substance. Thus, the invention provides means for the detection of specific signals on a microarray.

In yet another embodiment of the invention, double-stranded hybrids as formed on a microarray could be detected by the intercalation of a double-strand-specific dye. To confirm the specificity of the signals on the microarray, stretches of double-stranded DNA which are labeled by the dye could be subjected to digestion by means of a double-strand-specific endonuclease. In a preferred embodiment of the invention, the double-strand-specific endonuclease is DSN. In a further preferable embodiment of the invention, the double-strand-specific endonuclease is used in the presence of a single-stranded-DNA binding substance. In an even more preferable embodiment of the invention, the double-strand-specific endonuclease is DSN, which is used in the presence of a single-stranded-DNA binding substance. Thus, the invention provides just another means for the detection of specific signals on a microarray.

Single-stranded DNA is further used in hybridization experiments like the preparation of testers and drivers for subtractive hybridizations during the preparation of a plurality of nucleic acid molecules. Such technologies, as known to a person skilled in the art, are further described by C. G. Sagerström et al., Ann. Rev. Biochem. Vol. 66, 1997, pages 751 to 783, which is hereby incorporated herein by reference.

Normalization

In one such embodiment, the invention can be applied to the normalization of a sample, when the complexity of such a sample is reduced by the removal of highly repetitive sequences or by the removal of frequently occurring molecules having the same sequence. In this application, a given plurality of double-stranded nucleic acid molecules is denatured to separate the two strands from each other, for example, by heat treatment or any other method known to a person skilled in the art. After denaturation, single-stranded nucleic acid molecules within the sample are allowed to re-associate forming double-stranded nucleic acid molecules comprising two nucleic acid strands of complementary sequences. As the reassociation kinetics are directly dependent on the concentration of complementary nucleic acid molecules within the sample, nucleic acid molecules present in high or higher concentrations re-associate faster or much faster than nucleic acid molecules present in low or very low concentrations. Thus, after a given time, abundant nucleic acid molecules have formed preferentially double-stranded nucleic acid molecules whereas rare nucleic acid molecules are still present as single-stranded nucleic acid molecules. At a time point as selected or established to suite experimental needs, the hybridization reaction is terminated and the sample is treated with a double-strand-specific endonuclease in the presence or absence of a single-stranded-DNA binding substance to digest double-stranded DNA molecules which have been formed by the abundant nucleic acid molecules within the sample. Thus, the invention provides a means for the normalization of any plurality of nucleic acid molecules by means of an enzymatic activity removing specific hybrid molecules comprising partially or entirely of double-stranded nucleic acids.

Subtraction

In yet another embodiment, the invention can be applied to the subtraction from a sample which is called a tester. Certain nucleic acid molecules that are common to some of nucleic acid molecules in the tester are present in a so-called driver. Nucleic acid molecules common to the driver and the tester having highly related or the same sequence are entirely or partially removed or “subtracted” from the tester. In such an application, two given pluralities of single-stranded nucleic acid molecules as prepared by means of the invention or any other method known to a person skilled in the art are mixed to allow for the association of single-stranded nucleic acid molecules forming double-stranded nucleic acid molecules comprising two nucleic acid strands of complementary sequences. At a given time point, related nucleic acid molecules form preferentially double-stranded nucleic acid molecules, whereas nucleic acid molecules for which no complementary nucleic acid molecules are present in the driver remain as single-stranded nucleic acid molecules. At a time point selected or established to suite experimental needs, the hybridization reaction is terminated and the sample is treated with a double-strand-specific endonuclease in the presence or absence of a single-stranded-DNA binding substance to digest double-stranded DNA molecules which correspond to nucleic acid molecules common to the tester and driver. Thus, the invention provides a means for the subtraction of any plurality of nucleic acid molecules with a driver by means of an enzymatic activity removing specific hybrid molecules comprising partially or entirely of double-stranded nucleic acids.

In another embodiment, the invention relates to the normalization and subtraction of any plurality of nucleic acid molecules in which the nucleic acid molecules within the plurality of nucleic acid molecules can include ribonucleic acid molecules or deoxyribonucleic acid molecules in any possible combination including homodimers and heterodimers thereof.

Furthermore, the invention relates to the detection and measurement of single-stranded DNA in the presence of double-stranded DNA, which an aliquot of such a plurality of nucleic acids including any of single-stranded DNA, partly single-stranded and partly double-stranded DNA and double-stranded DNA, is taken out from the plurality of nucleic acids and subjected to the digestion of the double-stranded DNA by a double-strand-specific endonuclease. Such a reaction can be performed by means of the double-strand-specific endonuclease only or by the combined use of the double-strand-specific endonuclease and a single-stranded-DNA binding substance. As the enzymatic activity will digest the double-stranded DNA within the plurality of nucleic acids, single-stranded DNA remains in solution and can be subject to detection and measurement by methods known to a person skilled in the art. Such an approach includes, but is not limited to, the use of OliGreen®, a sensitive fluorescent nucleic acid stain, commercially available from Molecular Probes, Eugene, USA (Cat. No. O-7582 and O-11492). As outlined above, OliGreen® does not detect short polynucleotides or single nucleotides as released into the reaction mixture after the digestion of the double-stranded DNA. Thus, the use of OliGreen® allows for the direct measurement of the reaction products in which single-stranded DNA is of primary interest, without the need to remove short polynucleotides or single nucleotides from the reaction mixture. Alternative approaches can further make use of a radioactive label as incorporated into the DNA template and as applied to the digestion of double-stranded DNA, and in which amounts of single-stranded DNA obtained from such a reaction mixture is analyzed for the amount of radioactive label incorporated into the sample. Furthermore the reaction products can be analyzed by gel electrophoreses and staining of the reaction products applying standard technologies known to a person skilled in the art or described by J. Sambrook and D. W. Russell, ibid.

Thus, any such application of single-stranded DNA as prepared by the methods disclosed herein is within the scope of the invention, and the invention provides the necessary means to prepare the single-stranded DNA to be used in any such application.

DNA-RNA Hybrids

In another embodiment, the invention relates to the digestion of the DNA strand in double-stranded hybrid molecules composed of a RNA strand and a DNA strand by means of a double-strand-specific endonuclease. In any plurality of nucleic acids encompassing single-stranded DNA, double-stranded DNA, single-stranded RNA, double-stranded RNA, and hybrid molecules composed of RNA-DNA hybrids including hybrids composed entirely of double-stranded RNA-DNA hybrids or containing regions of partially single-stranded RNA or single-stranded DNA beside double-stranded RNA-DNA hybrids, a double-strand-specific endonuclease can be applied to digest the DNA strand as part of the RNA-DNA hybrid. Such a reaction can be performed by means of the double-strand-specific endonuclease only or by the combined use of the double-strand-specific endonuclease in combination with a single-stranded-DNA binding substance.

More precisely, the invention relates to the isolation of RNA from any plurality of nucleic acids including any of single-stranded DNA, double-stranded DNA, single-stranded RNA, and hybrid molecules composed of RNA-DNA hybrids including hybrids composed entirely of double-stranded RNA-DNA hybrids or containing regions of partially single-stranded RNA or DNA beside double-stranded RNA-DNA hybrids by means of a double-strand-specific endonuclease, in which such a reaction can be performed by means of the double-strand-specific endonuclease only or by the combined use of the double-strand-specific endonuclease and a single-stranded-DNA binding substance. The single-stranded DNA from such a reaction mixture can be digested by means of an exonuclease as known to a person skilled in the art or as described by J. Sambrook and D. W. Russell, ibid. The remaining RNA can then be isolated, analyzed, and cloned by standard methods as known to a person skilled in the art or as described by J. Sambrook and D. W. Russell, ibid.

Even more precisely, the invention relates to the isolation of single-stranded DNA from any plurality of nucleic acids including any of single-stranded DNA, double-stranded DNA, single-stranded RNA, and hybrid molecules composed of RNA-DNA hybrids including hybrids composed entirely of double-stranded RNA-DNA hybrids or containing regions of partially single-stranded RNA or DNA beside double-stranded RNA-DNA hybrids by means of a double-strand-specific endonuclease, where such a reaction can be performed by means of the double-strand-specific endonuclease only or by the combined use of the double-strand-specific endonuclease in combination with a single-stranded-DNA binding substance. RNA from such a reaction mixture can be digested by means of a ribonuclease as known to a person skilled in the art or as described by J. Sambrook and D. W. Russell, ibid. The remaining single-stranded DNA can then be isolated, analyzed and cloned by standard methods known to a person skilled in the art or described by J. Sambrook and D. W. Russell, ibid.

Furthermore, the invention relates to methods to clone single-stranded DNA from pluralities of nucleic acids composed of partly single-stranded and partly double-stranded DNA or DNA/RNA hybrids. In this embodiment of the invention, the double-stranded DNA or DNA/RNA hybrids in such a plurality of nucleic acids are digested by means of a double-strand-specific endonuclease. In a preferable embodiment of the invention, the double-strand-specific endonuclease is used for an independent enzymatic activity. In an even more preferable embodiment of the invention the double-strand-specific endonuclease is used in combination with a single-stranded-DNA binding substance. The single-stranded DNA recovered from the reaction mixture can be isolated and cloned by standard methods known to a person skilled in the art. In this embodiment, the invention relates to the cloning of genomic regions from genomic DNA, in which the genomes comprise partially or entirely single-stranded DNA. In a more preferable embodiment, the invention relates to the cloning of any single-stranded DNA from a plurality of nucleic acids, in which double-stranded nucleic acid molecules are digested by means of a double-strand-specific endonuclease. In an even further preferable embodiment, the invention relates to the cloning of any single-stranded DNA from a plurality of nucleic acids. Double-stranded nucleic acid molecules are digested by means of a double-strand-specific endonuclease in the presence of a single-stranded-DNA binding substance. Thus, the invention in general relates to methods for the cloning of single-stranded DNA.

Even further, the invention relates to a method involving or depending on the preparation of single-stranded DNA by means of a double-strand-specific endonuclease together with or without an additional single-stranded-DNA binding substance as disclosed herein, and the use thereof to develop, manufacture, market, sale, use or apply a kit, which includes any such enzymatic activities or allows for the use of such enzymatic activities for commercial reasons. Thus, the invention encompasses the use of any enzymatic activity of a double-strand-specific endonuclease together with or without an additional single-stranded-DNA binding substance as disclosed herein for commercial use in service, production, and manufacturing.

Seeing the wide range of applications for single-stranded DNA in the field of biotechnology and molecular biology in general, the invention as disclosed herein, will be of great commercial value in offering novel means for the preparation and application of single-stranded DNA for reagents, kits, and services in research, biotechnology, and diagnostic markets.

### EXAMPLES

The present invention will now be further explained in more detail with reference to the following examples. All names and abbreviations as used to describe the invention herein shall have the meaning as known to a person skilled in the art.

### Example 1

Template DNA for the preparation of single-stranded DNA can be circular or linear RNA or DNA, already single-stranded or double-stranded by nature, and can be obtained, prepared or modified by any method known to a person skilled in the art. Thus, the invention is not limited to the use of a particular source of DNA or RNA.

For the purpose of this example, a cDNA library prepared from a melanoma cell culture and cloned into the vector system Lambda-FLC, disclosed in patent application PCT/JP02/01667, which is hereby incorporated herein by reference, was used to perform the invention. From an aliquot of the aforementioned cDNA library, plasmid DNA was isolated by standard protocols as described by P. Carninci et al. in Genomics Vol. 77, 2001, pages 79-90, which is hereby incorporated herein by reference. The plurality of plasmid DNA obtained was characterized by digestion with the restriction endonuclease PvuII to measure the size of the cDNA inserts by gel electrophoresis. To perform the invention, the plurality of the plasmid DNA comprising the entire cDNA library or individual clones derived thereof were used as disclosed below. For the individual reactions as disclosed herein, it is not relevant whether DNA from an individual clone or DNA samples comprising the entire cDNA library were applied to perform the invention. Thus, individual reactions do not depend on the nature of the DNA used, but may have to be adjusted depending on the amounts of DNA present in a given reaction mixture.

Plasmid DNA from individual clones or the entire cDNA library was transformed and amplified in the bacterial strain DH10B, Invitrogen, Carlsbad, USA, and plasmid DNA as use for the examples was purified from bacterial cultures grown in LB Medium (J. Sambrook and D. W. Russell, ibid) by the use of a Qiagen QIAprep Spin Miniprep Kit for plasmid DNA isolation (Qiagen, Hilden, Germany, Cat. No. 27104).

For the preparation of circular single-stranded DNA from double-stranded plasmid DNA, the GeneTrapper® cDNA Positive Selection System from Gibco BRL/Life Technologies (CAT. NO. 10356-020, nowadays part of Invitrogen Corporation, Carlsbad, USA) was applied according to the maker's instruction, and the Instruction Manual of which is hereby incorporated herein by reference.

In one embodiment of the invention, plasmid DNA from a randomly isolated cDNA clone derived from the aforementioned cDNA library was used for a better validation of the experimental conditions. In brief, in total volume of 20 μl or 1 μl of a Gene II enzyme solution, as provided in the kit, was applied to the 5 μg of the plasmid DNA in a IXGeneII reaction buffer, as provided in the kit. The reaction mixture was incubated for 45 min at 30° C. in a water bath, before the reaction was terminated at 65° C. for 5 min, followed by immediately placing the sample on ice. The nicked DNA was then subjected to treatment with ExoIII as provided in the kit. To 19 μl of the aforementioned reaction mixture 2 μl of the ExoIII enzyme solution, as provided in the kit, were added, and the reaction mixture was further incubated at 37° C. for 1 hr. After the incubation, 20 μl of the reaction mixture was extracted with the same volume of phenol:chloroform. The aqueous phase was re-extracted with chloroform before adding 0.5 μl of 5M NaCl and 50 μl of absolute ethanol for the precipitation of DNA from the supernatant. After incubation at minus 20° C. for 30 min, DNA was collected by centrifuged at 15,000 rpm for 15 min. The pellet was washed twice with 80% ethanol and obtained by centrifugation as described above. Finally the DNA was dissolved in 50 μl of water.

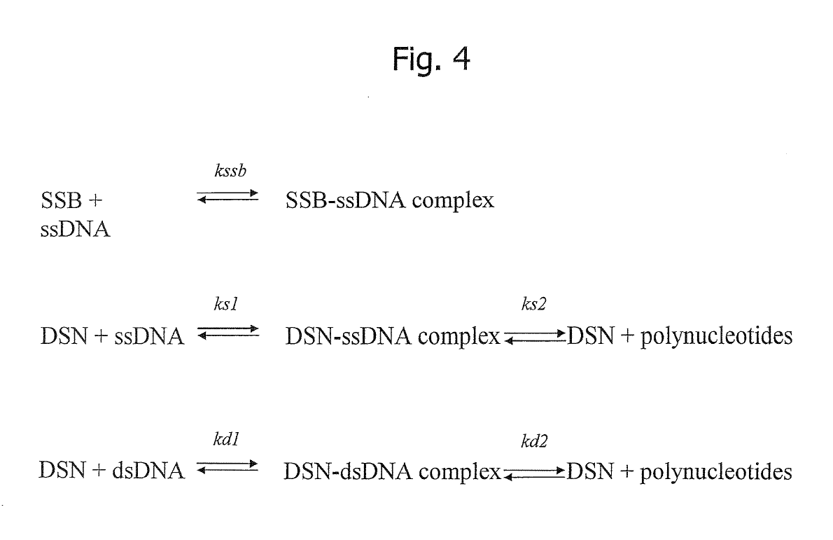

Throughout the preparation aliquots of 1 μl were taken at each step to monitor the progress of the preparation by gel electrophoresis. The aforementioned reaction was monitored by loading control samples from each step on a 0.8% agarose gel, and analyzed in the presence of untreated vector as a control. The agarose gel electrophoresis was performed as described by J. Sambrook and D. W. Russell, ibid, and DNA was visualized by staining with SYBR Green II (BioWhittaker Molecular Applications, Rockland, USA, Part Code 50523). One example of such an experiment is shown in FIG. 4. Only samples, for which the agarose gel analysis showed distinct changes in the patterns in comparison to the control, were subjected to further treatment. Although agarose gel electrophoresis allows for the distinction between supercoiled, relaxed, and single-stranded DNA, often background of double-stranded DNA is visible at the same time.

In order to remove double-stranded DNA from preparations of single-stranded DNA, additional purification steps were performed. The aforementioned reaction mixture was first incubated at 65° C. for 5 min to dissociate secondary structures which may have formed in the single-stranded DNA molecules. After heat treatment, the sample was immediately placed on ice. For digestion with DSN, 20 μl of the sample were mixed with 2.5 μl of 10XDSN buffer (Evrogen, Cat.# EA001, Moscow, Russia) and 1 μl of the single-stranded-DNA binding protein T4gene-32 (4.5 μg/μl, USB, Cleveland, USA). After the solution was adjusted to a final volume of 24 μl with water, 1 μl of a DSN enzyme stock (1 unit per μl, Evrogen, Cat.# EA001, Moscow, Russia) was added and the reaction mixture was incubated at 37° C. for 1 hr. Addition of 1 μl of a 0.5M EDTA stock solution terminated the reaction, before the volume was adjusted with water to 50 μl, and 1 μl of 10% of SDS was added. Remaining DSN activity was destroyed by Proteinase K treatment, for which 2 μl of a Proteinase K enzyme solution (20 μg/μl, Qiagen, Hilden, Germany, Cat. NO. 19131) were added, and the reaction mixture was incubated at 45° C. for 2 hrs. After the Proteinase K treatment the reaction mixture was extracted with equal volumes of phenol:chloroform and chloroform under standard conditions. Single-stranded DNA was precipitated out of the aqueous phase by adding 1 μl of a 2 μg/μl glycogen solution, 2.5 μl of 5M NaCl, and 150 μl of absolute ethanol. After incubation at minus 20° C. for 30 min, DNA was collected by centrifugation as described above. The DNA pellet was washed twice with 80% ethanol before the DNA was finally dissolved in 40 μl of water.

In order to remove linear single-stranded DNA from circular single-stranded DNA, the sample was further incubated with an exonuclease. Out of the aforementioned DNA preparation, a 40 μl sample was mixed with 5 μl ExoI 10X reaction buffer (New England Biolabs® Inc, Beverly, USA) and 1 μl of an ExoI enzyme solution (2 units/μl, New England Biolabs® Inc, Beverly, USA, Cat. No. N0293S) to obtain a final volume of 50 μl. After incubation at 37° C. for 1 h, the reaction was terminated by Proteinase K treatment as described above, and circular single-stranded DNA was isolated after phenol:chlorophorm extraction by ethanol precipitation. The DNA was finally dissolved in 20 μl of water.

### Example 2

Activity of DSN against single-stranded DNA was tested by the use of radioactively labeled single-stranded DNA prepared from the aforementioned library G2 as a sample. Sample DNA was labeled with □P32-GTP (Amersham Biosciences, Cardiff, United Kingdom) as described in “DNA Micorarrays: A Molecular Cloning Manual”, edited by D. Bowtell et al., Cold Spring Harbor Laboratory Press, 2003, which is hereby incorporated herein by reference. In this example the effect of different single-stranded-DNA binding substances was tested, and they were compared for the effect on DSN activity as well as the protection of the single-stranded DNA. To perform the experiment, the radioactive sample was divided into four equal aliquots each of which contained 250 ng of single-stranded DNA in 7 μl of water plus 1 μl of 10x DSN Buffer (Evrogen, Cat.# EA001, Moscow, Russia). After heat treatment of the samples at 65° C. for 5 min, the following reactions were performed in a final volume 10 μl: First, Control sample with no further additions, Second: plus 1 μl of 0.25 unit of DSN (Evrogen, Cat.# EA001, Moscow, Russia), Third: plus 1 μl of 0.25 unit of DSN (Evrogen, Cat.# EA001, Moscow, Russia) plus 1 μl of T4-gene 32 protein (USB, Cleveland, USA, Cat. No. 74029Y), and Forth: plus 1 μl of 0.25 unit of DSN (Evrogen, Cat.# EA001, Moscow, Russia) plus 1 μl of E.coli protein SSB (Promega, Madison, USA, Cat. No. M3011). After incubation on 37° C. for 1 h, 1 μl of 0.5M EDTA was added to all samples to terminate the reactions before the samples were mixed with 3 μl of alkaline loading buffer for gel electrophoresis (300 mM NaOH, 30 mM EDTA, 30% glycerol, 0.2% Brome Phenol Blue). Then samples were loaded on 0.8% alkaline agarose gel (Dojindo, Mashiki, Japan, Cat. No. 344-00073) and run in 1 X TBE buffer for 10 h at 25V. As a P³² labeled Lambda/HindIII marker was applied (New England Biolabs® Inc, Beverly, USA, Cat. No. N3012S), samples could be directly visualized by autoradiography. The following day the gel was washed with 10% acetic acid and dried on a gel dryer (Bio-Rad, Hercules, USA). The dried gel was exposed for about 1.5 h to image the radioactive samples, and a BAS-5000 image analyzer (Fuji film, Tokyo, Japan) was used to further analyze the signals. The experiment as presented in this example demonstrated that different single-stranded-DNA binding substances could be used in their own right to perform the invention.

### Example 3

Activity of DSN against linear single-stranded DNA was tested by the use of radioactively labeled liner single-stranded DNA prepared from the aforementioned G2 mRNA sample. Linear single-stranded DNA was synthesized and labeled with □P32-GTP (Amersham Biosciences, Cardiff, United Kingdom) as described in “DNA Micorarrays: A Molecular Cloning Manual”, edited by D. Bowtell et al., Cold Spring Harbor Laboratory Press, 2003, which is hereby incorporated herein by reference. After heat treatment of the samples at 65° C. for 5 min, reactions were performed as disclosed in Example 2 using 0.25 unit of DSN (Evrogen, Cat.# EA001, Moscow, Russia), and 1 μl of E.coli protein SSB (Promega, Madison, USA, Cat. No. M3011) were indicated. After incubation at 37° C. or 65° C. for 1 h, the reactions were terminated and the samples were mixed with 3 μl of alkaline loading buffer for gel electrophoresis (300 mM NaOH, 30 mM EDTA, 30% glycerol, 0.2% Brome Phenol Blue). Afterwards samples were loaded on 0.8% alkaline agarose gel (Dojindo, Mashiki, Japan, Cat. No. 344-00073) and run in 1 X TBE buffer for 10 h at 25V. The following day the gel was washed with 10% acetic acid and dried on a gel dryer (Bio-Rad, Hercules, USA). The dried gel was exposed for about 1.5 h to image the radioactive samples on a BAS-5000 image analyzer (Fuji film, Tokyo, Japan). The experiment as presented in this example demonstrated that the specificity of DSN is temperature dependent in the absence of a single-stranded-DNA binding protein. However, the addition of SSB allowed for a specific digestion of double-stranded DNA in the presence of single-stranded DNA at any tempature between 37° C. and 65° C.

1. A method for purifying a single-stranded DNA from a mixture of the single-stranded DNA and a partially or entirely double-stranded DNA and/or DNA-RNA hybrid, comprising the steps of:
having a single-stranded-DNA binding substance which binds to single-stranded DNA molecules attached to the single-stranded DNA molecules so as to protect them,
digesting double-stranded DNA molecules by means of a double-strand-specific endonuclease which specifically cleaves double-stranded DNA and/or DNA-RNA molecules and which does not cleave single-stranded DNA to produce a digestion product, and
separating the protected single-stranded DNA molecules from the digestion product.

2. The method according to claim 1, wherein the single-stranded-DNA binding substance disrupts secondary structures the single-stranded DNA molecules may have.

3. The method according to claim 1, wherein the single-stranded DNA is subsequently separated from the protected single-stranded DNA molecules.

4. The method according to claim 1, wherein the single-stranded-DNA binding substance and the double-strand-specific endonuclease are used in combination to remove the DNA strand from a hybrid molecule composed of one strand of RNA and one strand of DNA in a plurality of nucleic acids comprised of RNA and DNA.

5. The method according to claim 1, wherein the single-strand-specific DNA binding substance is a protein.

6. The method according to claim 5, wherein the single-strand-specific DNA binding substance is an antibody against the single-stranded DNA.

7. The method according to claim 1, wherein the single-stranded-DNA binding substance is a protein selected from a group consisting of SSB obtainable from E. coli, a product of phage T4 Gene 32, adenovirus DBP, an antibody directed against the single-stranded DNA molecules, calf thymus UP1, and any mixture thereof.

8. The method according to claim 1, wherein the double-strand-specific endonuclease is Duplex-Specific Nuclease obtainable from crab hepatopancreas or a mixture of four-base-pair cutters which are restriction endonucleases having a recognition site comprising four constitutive nucleotides within a double-stranded DNA molecule.

9. A method for preparing a circular single-stranded DNA from a circular double-stranded DNA or a mixture of circular single- and double-stranded DNAs comprising the steps of:
cutting a circular double-stranded DNA with an enzyme that has nicking activity to introduce a cut into one of the two DNA strands making up the circular double-stranded DNA,
digesting the one strand cut by the nicking enzyme with an exonuclease to produce circular single-stranded DNA molecules,
having a single-stranded-DNA binding substance which specifically binds to single-stranded DNA molecules attached to the single stranded DNA molecules so as to protect them,
digesting remaining double-stranded DNA molecules by means of a double-strand-specific endonuclease which specifically cleaves double-stranded DNA molecules, and
removing linear single-stranded DNA molecules from circular single-stranded DNA molecules by means of an exonuclease.

10. The method according to claim 9, wherein the combined use of a single-stranded-DNA binding substance and a double-strand-specific endonuclease removes double-stranded DNA from a preparation of linear or circular single-stranded DNA.

11. The method according to claim 9, wherein a single-stranded-DNA binding substance and a double-strand-specific endonuclease are used in combination to remove the DNA strand from a hybrid molecule composed of one strand of RNA and one strand of DNA in a plurality of nucleic acids comprised of RNA and DNA.

12. The method according to claim 9, wherein the single-strand-specific DNA binding substance is a protein.

13. The method according to claim 12, wherein the single-strand-specific DNA binding substance is an antibody against the single-stranded DNA.

14. The method according to claim 9, wherein the single-stranded-DNA binding substance is a protein selected from a group consisting of SSB obtainable from E. coli, a product of phage T4 Gene 32, adenovirus DBP, an antibody directed against the single-stranded DNA molecules, calf thymus UP1, and any mixture thereof.

15. The method according to claim 9, wherein the double-strand-specific endonuclease is Duplex-Specific Nuclease obtained from crab hepatopancreas or a mixture of four-base-pair cutters which are restriction endonucleases having a recognition site comprising four constitutive nucleotides within a double-stranded DNA molecule.

16. A method for removing double-stranded DNA molecules from a mixture of double-stranded and single-stranded DNA molecules, comprising the steps of:
adding to the mixture a single-stranded-DNA binding substance which binds to single-stranded DNA molecules, and
adding to the mixture a double-strand-specific endonulease which specifically cleaves double-stranded DNA molecules.

17. The method according to claim 16, wherein the single-stranded-DNA binding substance is a protein.

18. The method according to claim 17, wherein the single-strand-specific DNA binding substance is an antibody against the single-stranded DNA.

19. The method according to claim 16, wherein the single-stranded-DNA binding substance is a protein selected from a group consisting of SSB obtainable from E. coli, a product of phage T4 Gene 32, adenovirus DBP, an antibody directed against the single-stranded DNA molecules, calf thymus UP1, or any mixture thereof.

20. The method according to claim 16, wherein the double-strand-specific endonuclease is Duplex-Specific Nuclease obtained from crab hepatopancreas or a mixture of four-base-pair cutters which are restriction endonucleases having a recognition site comprising four constitutive nucleotides within a double-stranded DNA molecule.

21. A method for removing nucleic acid molecules having a certain nucleic acid sequence and belonging to a tester, comprising the steps of:
preparing driver oligonuleotide molecules that can hybridize to the nucleic acid molecules having the certain nucleic acid sequence and belonging to the tester,
adding the driver oligonuleotide molecules in an excess amount compared to the amount of the nucleic acid molecules having the certain nucleic acid sequence to a sample containing the tester, and
adding a double-strand-specific endonulease to the sample so as to digest double-stranded molecules formed between the driver oligonucleotide molecules and the nucleic acid molecules having the certain nucleic acid sequence.

22. The method according to claim 21, wherein the sample is subsequently subjected to the steps of:
removing the double-strand-specific endonulease from the sample, and
amplifying nucleic acid molecules that belong to the tester and remain in the sample.

23. The method according to claim 21, wherein a single-stranded DNA binding substance is added while digesting with a double-strand-specific endonuclease.

24. The method according to claim 21, wherein the single-stranded-DNA binding substance is a protein.

25. The method according to claim 24, wherein the single-strand-specific DNA binding substance is an antibody against the single-stranded DNA.

26. The method according to claim 21, wherein the single-stranded-DNA binding substance is a protein selected from a group consisting of SSB obtainable from E. coli, a product of phage T4 Gene 32, adenovirus DBP, an antibody directed against the single-stranded DNA molecules, calf thymus UP1, and any mixture thereof.

27. The method according to claim 21, wherein the double-strand-specific endonuclease is Duplex-Specific Nuclease obtained from crab hepatopancreas or a mixture of four-base-pair cutters which are restriction endonucleases having a recognition site comprising four constitutive nucleotides within a double-stranded DNA molecule.

28. A kit for purifying a single-stranded DNA from a mixture of the single-stranded DNA and a partially or entirely double-stranded DNA and/or DNA-RNA hybrid, comprising:
a single-stranded-DNA binding substance which binds to single-stranded DNA molecules so as to protect them, and
a double-strand-specific endonuclease which specifically cleaves double-stranded DNA molecules and which does not cleave single-stranded DNA to produce a digestion product.

29. The kit according to claim 28, wherein the single-strand-specific DNA binding substance is a protein.

30. The kit according to claim 28, wherein the single-strand-specific DNA binding substance is an antibody against the single-stranded DNA.

31. The kit according to claim 28, wherein the single-stranded-DNA binding substance is a protein selected from a group consisting of SSB obtainable from E. coli, a product of phage T4 Gene 32, adenovirus DBP, an antibody directed against the single-stranded DNA molecules, calf thymus UP1, or any mixture thereof.

32. The kit according to claim 28, wherein the double-strand-specific endonuclease is Duplex-Specific Nuclease obtained from crab hepatopancreas or a mixture of four-base-pair cutters which are restriction endonucleases having a recognition site comprising four constitutive nucleotides within a double-stranded DNA molecule.

33. A kit for preparing a circular single-stranded DNA from a circular double-stranded DNA or a mixture of circular single- and double-stranded DNAs comprising:
an enzyme that has nicking activity to introduce a cut into one of the two DNA strands making up the circular double-stranded DNA,
an exonuclease that digests the nicked strand for producing circular single-stranded DNA molecules,
a single-stranded-DNA binding substance which binds to single-stranded DNA molecules so as to protect them, and
a double-strand-specific endonuclease which specifically cleaves double-stranded DNA molecules and which does not cleave single-stranded DNA to produce a digestion product.

34. The kit according to claim 33, wherein the single-strand-specific DNA binding substance is a protein.

35. The kit according to claim 34, wherein the single-strand-specific DNA binding substance is an antibody against the single-stranded DNA.

36. The kit according to claim 33, wherein the single-stranded-DNA binding substance is a protein selected from a group consisting of SSB obtainable from E. coli, a product of phage T4 Gene 32, adenovirus DBP, an antibody directed against the single-stranded DNA molecules, calf thymus UP1, and any mixture thereof.

37. The kit according to claim 33, wherein the double-strand-specific endonuclease is Duplex-Specific Nuclease obtained from crab hepatopancreas or a mixture of four-base-pair cutters which are restriction endonucleases having a recognition site comprising four constitutive nucleotides within a double-stranded DNA molecule.
